(19) United States
(12) Patent Application Publication
(10) Pub. No.: US 2025/0387170 A1
(43) Pub. Date: Dec. 25, 2025

(54) MONITORING DEVICE FOR NAVIGATION-GUIDED MEDICAL INTERVENTIONS

(71) Applicant: Quantum Surgical, Montpellier (FR)

(72) Inventors: Yann LE MEUR, Saint-Brès (FR); Barnabé SIMON, Montpellier (FR); Lucien BLONDEL, Montpellier (FR); Bertin NAHUM, Castelnau-le-Lez (FR); Fernand BADANO, Lyon (FR)

(21) Appl. No.: 18/880,208

(22) PCT Filed: Jun. 19, 2023
(86) PCT No.: PCT/EP2023/066498

§ 371 Date: Dec. 30, 2024

(30) Foreign Application Priority Data
Jun. 29, 2022 (FR) FR2206575

(51) Int. Cl. A61B 34/20; A61B 90/00

(52) U.S. Cl. CPC A61B 34/20; A61B 2034/2055; A61B 2034/2061; A61B 2034/2072; A61B 2090/3966

(57) ABSTRACT

The invention relates to an optical monitoring device for monitoring the movements of an anatomical part of interest of a patient during a minimally invasive medical intervention. The optical monitoring device comprises a base layer that serves as a sterile drape and comprises an intervention region and a marking region which at least partially surrounds the intervention region. The marking region comprises, on the inner face, an adhesive material for attaching the monitoring device to the skin of the patient and, on the outer face, at least three optical markers or at least three attachment supports each intended to accommodate an optical marker. The marking region also comprises an optical fibre sensor which is securely attached to the base layer and which has a measurement point associated with each of the optical markers or attachment supports.

### FIELD OF THE INVENTION

The devices and the methods disclosed in the present application belong to the field of optical navigation to follow the movements of an anatomy of interest of a patient during a minimally-invasive medical intervention. There is more particularly proposed an optical monitoring device based on the skin of the patient and an optical navigation method utilizing such an optical monitoring device.

### PRIOR ART

Minimally-invasive medical interventions necessitate very precise positioning or movement of a medical instrument (for example a needle, a catheter, an electrode, an ultrasound generator, a drill, etc.) relative to an anatomy of interest of a patient (for example the liver, a lung, a kidney, a bone, etc.). The practitioner who carries out this type of intervention may be assisted by a medical robot. In this case the medical robot positions, holds and/or guides a medical instrument relative to the anatomy of interest thanks to a navigation system. The instrument is for example fixed to one end of an articulated arm of the robot. The navigation system enables determination of the position of the instrument and the position of the anatomy of interest. Information on the respective positions of the instrument and the anatomy of interest relative to one another enable the robot to configure its articulated arm so that the instrument is positioned optimally relative to the anatomy of interest.

In the case of an optical navigation system an optical monitoring device is generally placed on the skin of the patient near the anatomy of interest. This monitoring device generally includes at least three optical markers to enable the navigation system to determine precisely the position of the monitoring device. The position of the anatomy of interest can then be determined on the basis of the position of the monitoring device and with the aid of a medical image in which can be seen both the anatomy of interest and the monitoring device (or at least a part of the monitoring device).

During the intervention the anatomy of interest may move, for example because of respiratory movements of the patient. It is therefore necessary to be able to monitor the position of the anatomy of interest over time with the aid of the navigation system. However, the line of sight between an optical marker of the monitoring device and an optical sensor of the navigation system may be interrupted by an obstacle (for example by the practitioner, by the instrument or by the articulated arm of the robot), and this can impede the determination of the position of the monitoring device.

The various optical markers are sometimes stuck individually onto the skin of the patient. Apart from the fact that this is a relatively lengthy and complex process, the adhesion of the optical markers to the skin of the patient is sometimes insufficient and they may become unstuck because of inadvertence by the practitioner during the intervention. In some cases the optical markers are all fixed to the same adhesive tape intended to be stuck to the skin of the patient to surround the intervention zone. In all cases the optical markers must not be masked by the sterile drape that covers the patient. It is therefore necessary to cut a large opening or a plurality of openings at different places in the sterile drape, which is not desirable because it is necessary to disinfect the skin of the patient at the level of these opening zones.

The patent application US 2020/0008897 A1 describes a monitoring device comprising a base layer serving as a sterile drape, a plurality of optical markers fixed to the exterior face of the base layer and an adhesive material on the interior face for sticking the base layer to the skin of the patient. During the intervention an incision is made on the patient through the base layer of the monitoring device (in other words, the base layer and the skin of the patient must be incised at the same time because all of the surface of the base layer is adhesive). However, this type of device is not suitable for percutaneous interventions. In fact, for a percutaneous intervention the principle is to insert needles directly through the skin of the patient, with no previous incision, in order to reduce the risks of contamination of the patient. It is also frequently the case in this type of intervention to treat a plurality of lesions, or to treat a lesion with the aid of a plurality of needles via different paths. Thus the simultaneous incision in the monitoring device and the patient cannot be done in the context of this minimally-invasive practice.

### STATE OF INVENTION

The solutions proposed in the present application have the objective of remedying some or all of the drawbacks of the prior art, in particular those disclosed hereinabove.

To this end and in accordance with a first aspect, there is proposed an optical monitoring device for monitoring the movements of an anatomy of interest of a patient during a minimally-invasive medical intervention. The optical monitoring device comprises a base layer that serves as a sterile drape and has an interior face intended to face the skin of the patient and an exterior face opposite the interior face. The base layer comprises an intervention region corresponding to an opening or to a region intended to be cut to expose a zone of the skin of the patient where the intervention is to take place. The base layer also comprises a marking region that surrounds at least partially the intervention region. The marking region includes on the interior face an adhesive material for fixing the optical monitoring device onto the skin of the patient. The marking region includes on the exterior face at least three optical markers or at least three fixing supports each intended to receive an optical marker. The marking region also includes a fiber optic sensor fastened to the base layer and including at least one measuring point associated with each of the optical markers or the fixing supports.

The optical monitoring device is particularly easy to install on the patient because all of the optical markers or all of the fixing supports for the optical markers are fastened together. It suffices to apply the adhesive material to the skin of the patient to install the optical monitoring device.

The optical monitoring device intrudes minimally on the intervention zone. The extended configuration of the monitoring device with a localized distribution of the optical markers in fact allows the practitioner unimpeded access to the intervention zone.

The configuration of the monitoring device enables its adhesion to the skin of the patient to be optimized, which enables limitation of the risk of involuntary movement of an optical marker during the intervention. The adhesion area of the adhesive material can in fact be optimized thanks to the a priori knowledge of the location of the optical markers on the monitoring device.

The monitoring device also makes it possible to reduce the area of the skin of the patient to be disinfected. In fact, the monitoring device integrates both the sterile drape and the optical markers (or the fixing supports for the optical markers) and the area to be disinfected corresponds only to the opening in the sterile drape corresponding to the intervention region.

The fiber optic sensor enables information to be obtained as to the position in space of the optical markers relative to one another. Thus even if some optical markers are not visible to the locating device (for example if the line of sight between the locating device and an optical marker is interrupted by an obstacle), it remains possible to estimate the position of the optical markers that are not visible based on the position of at least one visible optical marker and the positions of the optical markers relative to one another. The fiber optic sensor may in particular include an optical fiber incorporating Bragg gratings.

In particular embodiments the optical monitoring device may further have one or more of the following features, separately or in all technically possible combinations.

In particular embodiments the base layer is a polyethylene film lined with a cellulose absorbent film.

In particular embodiments the intervention region is precut in the base layer.

In particular embodiments the base layer includes visual indications defining the marking region.

In particular embodiments the adhesive material takes the form of an adhesive tape connecting together the various points at which the optical markers or the fixing supports are situated. The adhesive tape enables a semi-rigid connection to be produced between the optical markers, which facilitates their installation and optimizes their stability.

In particular embodiments the adhesive tape is a polyethylene film coated with an acrylic adhesive or a rayon woven fabric covered with an acrylic adhesive.

In particular embodiments the monitoring device further includes at least three radio-opaque markers each rigidly connected to a respective optical marker or a respective fixing support. The radio-opaque markers are intended to identify the position of the optical monitoring device in a medical image.

In particular embodiments the optical markers are active, each active optical marker being configured to emit a differently modulated infrared signal.

In particular embodiments the optical markers are passive and the monitoring device includes at least four optical markers.

In accordance with a second aspect, there is proposed an optical navigation system including:
any of the above embodiments of an optical monitoring device, said optical monitoring device being equipped with optical markers,
a measuring device configured to cooperate with the fiber optic sensor to determine a relative position of each of the optical markers in a frame of reference of the measuring device,
a locating device configured to cooperate with the optical markers to determine a position of each of the optical markers in a frame of reference of the locating device.

In the present application an optical marker is considered “active” when it is configured to emit directly an optical signal without said signal having been generated by some other element. On the other hand, an optical marker is considered “passive” when it is configured to reflect an optical signal generated by another element.

In accordance with a third aspect there is proposed a navigation method utilizing an optical navigation system as described hereinabove. The method includes the following steps:
determination with the aid of the locating device of the position of at least one optical marker visible to the locating device,
identification of said at least one visible optical marker from among all the optical markers,
determination with the aid of the measuring device of the positions of all of the optical markers relative to one another,
determination of the position of at least one optical marker that is not visible to the locating device on the basis of the position of the visible optical marker and the positions of the optical markers relative to one another,
estimation of the position of the optical monitoring device from the positions of at least three optical markers.

In particular embodiments the optical markers are active, each optical marker is configured to emit a differently modulated infrared signal, and said at least one visible optical marker is identified by identifying the modulation of the infrared signal emitted by said optical marker.

In particular embodiments the optical markers are passive, the optical marking device includes at least four passive optical markers, the distances between two optical markers taken two by two all differ by at least one predetermined margin value, and the method further includes the following steps:
determination with the aid of the locating device of the position of at least three optical markers visible to the locating device,
identification of said three optical markers from among all of the optical markers on the basis of the distances between two optical markers determined for at least two different pairs of optical markers formed from among said at least three visible optical markers.

In particular embodiments the navigation method includes a preliminary step of generation for each optical marker with the aid of the locating device and the measuring device of a model representative of a substantially cyclic movement of said optical marker. The identification of said at least one visible optical marker is then effected by identifying the model corresponding to the movement of said visible optical marker.

### DESCRIPTION OF THE FIGURES

The invention will be better understood after reading the following description given by way of non-limiting example and with reference to FIGS. 1 to 4, which represent:

FIG. 1 a schematic representation of the exterior face of one exemplary embodiment of an optical monitoring device according to the invention,

FIG. 2 a schematic representation of the interior face of the optical monitoring device represented in FIG. 1,

FIG. 3 a schematic representation of one exemplary embodiment of an optical navigation system utilizing a monitoring device represented in FIGS. 1 and 2,

FIG. 4 a schematic representation of the main steps of an optical navigation method utilizing a navigation system represented in FIG. 3.

In these figures references that are identical from one figure to another designate identical or analogous elements. For the sake of clarity, the elements represented are not necessarily to the same scale, unless otherwise indicated.

### DETAILED DESCRIPTION OF ONE EMBODIMENT OF THE INVENTION

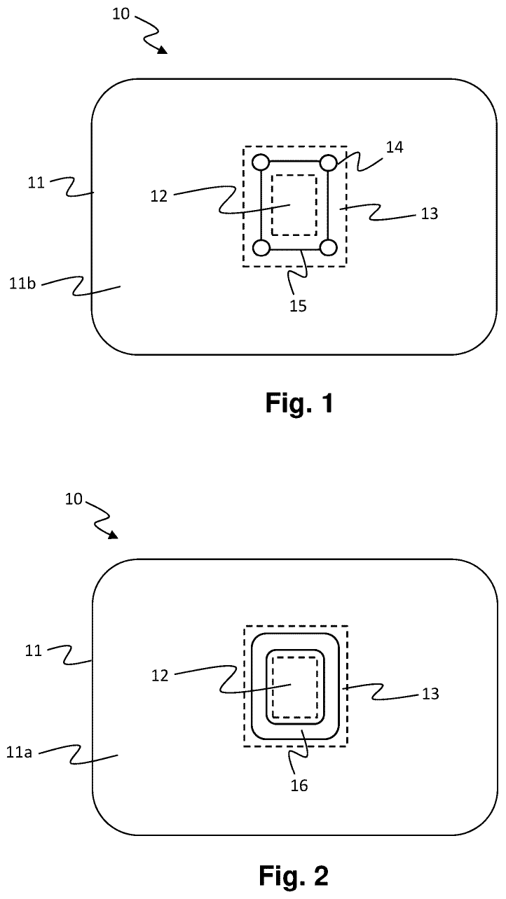

FIGS. 1 and 2 represent schematically one exemplary embodiment of an optical monitoring device 10 according to the invention.

The optical monitoring device 10 is intended to be installed on the skin of a patient near an anatomy of interest of the patient on which a minimally-invasive medical intervention has to be carried out.

The anatomy of interest corresponds for example to the liver, a lung, a kidney, a bone, etc. The minimally-invasive medical intervention aims for example to biopsy or to ablate a lesion in the anatomy of interest (for example a cyst, a tumor, etc.). The lesion may be ablated by various methods (radiofrequencies, microwaves, cryotherapy, laser, electroporation, focused ultrasound, etc.). It is then necessary to position or to move very precisely a medical instrument (for example a needle, a catheter, an electrode, an ultrasound generator, a drill, etc.) relative to the anatomy of interest of the patient. As described in detail hereinafter a locating device may be used to estimate the position of the optical monitoring device 10 in real time. A pre-intervention medical image that represents both the anatomy of interest of the patient and the optical monitoring device 10 can enable determination of the position of the anatomy of interest relative to the optical monitoring device 10. The real-time knowledge of the position of the optical monitoring device 10 can then enable determination in real time of the position of the anatomy of interest of the patient. The medical instrument can then be guided in real time as a function of the estimated position of the anatomy of interest of the patient. It should be noted that the position of the anatomy of interest and the position of the monitoring device 10 change over time, in particular because of respiratory movements of the patient.

In the present application the expression “the position of the monitoring device 10” must be understood in a broad sense, that is to say as encompassing both the position and the orientation of the monitoring device 10 (the word “pose” is sometimes used in the literature to represent the combination of the position and the orientation of an object). The same applies to the expression “the position of the medical instrument” which must be understood as “the position and the orientation of the medical instrument”, and the expression “the position of the anatomy of interest” which must be understood as “the position and the orientation of the anatomy of interest”.

As depicted in FIGS. 1 and 2 the optical monitoring device 10 comprises a base layer 11 that serves as a sterile drape. This base layer 11 has an interior face 11a and an exterior face 11b. The interior face 11a is represented in FIG. 2. The interior face 11a is intended to face the skin of the patient. The exterior face 11b is represented in FIG. 1. The exterior face 11b is opposite the interior face 11a.

The base layer 11 corresponds to a sterile drape. The base layer 11 includes for example a sheet of polyethylene lined with a cellulose absorbent film. The interior face 11a of the base layer 11 is then formed by the cellulose absorbent film. To reduce the risk of infection and cross-contamination the polyethylene film that forms the exterior face 11b of the base layer is preferably impermeable to bacteria and to liquids.

The base layer 11 comprises an intervention region 12 that corresponds to an opening or to a region intended to be cut in order to expose a zone of the skin of the patient where the intervention is to take place. In FIGS. 1 and 2 the intervention region 12 corresponds to the region situated inside the smaller dashed line rectangle.

In particular embodiments the intervention region 12 corresponds to a zone precut in the base layer 11. The part of the sterile drape forming the intervention region 12 can then easily be removed by the practitioner after installation of the optical monitoring device 10 on the patient. Alternatively the intervention region 12 can be cut by the practitioner and not precut. The size and the shape of the intervention region 12 are adapted to suit the envisaged medical intervention. The intervention region 12 may be situated at the center of the base layer 11 or elsewhere on the base layer 11.

The base layer 11 also comprises a marking region 13 that surrounds at least partially the intervention region 12. In FIGS. 1 and 2 the marking region 13 corresponds to the region situated between the two dashed line rectangles. For example and as depicted in FIGS. 1 and 2 the marking region 13 may take the form of a substantially rectangular strip around the intervention region. The marking region 13 could nevertheless also take some other form, such as a substantially circular strip, or a circular arc strip, or a C-shape, U-shape or V-shape strip that partially surrounds the intervention region 12.

As depicted in FIG. 2 the marking region 13 includes on the interior face 11a an adhesive material 16 for fixing the optical monitoring device 10 onto the skin of the patient.

As depicted in FIG. 1 the marking region 13 includes on the exterior face 11b at least three optical markers 14 or at least three fixing supports each intended to receive an optical marker 14.

An optical marker 14 corresponds for example to a reflecting sphere visible to an infrared stereoscopic camera (for example as in the Polaris® navigation solution from the company Northern Digital Inc.) or black and white patterns visible to a stereoscopic camera (for example as in the MicronTracker® navigation solution from ClaroNav). In these examples the optical markers are passive markers.

Alternatively the optical markers may be active markers configured to emit infrared signals detectable by an infrared camera. The optical markers may comprise retroreflecting lenses (such as the Radix® lenses developed by the company Northern Digital Inc. for example).

When the optical markers 14 are active it is advantageous for each optical marker 14 to be configured to emit a differently modulated infrared signal. Thus it is possible to identify each optical marker 14 from among all of the optical markers 14 on the basis of the infrared signal emitted by said optical marker 14.

It is advantageous to use optical markers of small size to limit the overall size of the optical monitoring device 10 and to facilitate access to the intervention zone.

The optical markers 14 are either integrated directly into the base layer 11 (they may for example be hot glued to the base layer 11) or mounted by an operator on the fixing supports of the optical monitoring device 10.

The position of the optical monitoring device 10 is determined on the basis of the position of at least three optical markers 14. The optical monitoring device 10 may advantageously include more than three optical markers 14 (or more than three fixing supports for the optical markers 14). This makes it possible to increase the chances of there being at least three optical markers 14 visible at a given time even if some optical markers are hidden by an obstacle.

The marking region 13 also includes a fiber optic sensor 15 fastened to the base layer 11. As described in detail hereinafter the fiber optic sensor 15 is intended to be connected to a measuring device to determine the position in space of the optical markers 14 relative to one another. To this end the fiber optic sensor 15 features at least one measurement point associated with each of the optical markers 14 or each of the fixing supports. The fiber optic sensor may be formed by a fiber incorporating Bragg gratings, as for example in the solution proposed by the company Sensuron® or by the company The Shape Sensing Company®. The fiber optic sensor 15 may be positioned equally well on the interior face 11a or the exterior face 11b of the base layer 11.

A Bragg grating is a resonant microstructure on the core of an optical fiber. This resonant structure acts as a mirror that is selective as a function of wavelength (narrowband filter around a wavelength specific to the Bragg grating): when light travels through the optical fiber, only a narrow part of the spectrum of the light centered on the wavelength of the Bragg grating is reflected. The rest of the spectrum of the light continues its path along the optical fiber to the next Bragg grating. The wavelength of a Bragg grating is essentially defined by the period of the microstructure and by the refractive index of the core of the fiber. The fiber can contain different Bragg gratings in series, each Bragg grating being associated with a specific wavelength. An opto-electronic measuring device is able to measure the wavelength reflected by each Bragg grating. Each Bragg grating corresponds to a measurement point. Deformation of the optical fiber leads to a change in the period of the microstructure and consequently also a change in the wavelength of the Bragg grating. It is therefore possible to determine a deformation applied to the optical fiber at the level of each Bragg grating by measuring a difference between a reference wavelength of the Bragg grating (the wavelength of the Bragg grating with no deformation) and a measured wavelength of the Bragg grating (the wavelength of the deformed Bragg grating). The measurement of the various deformations applied at the level of the various respective Bragg gratings enables determination of their positions in space relative to one another.

The marking region 13 integrates different technical elements. It is a technical zone that must not be degraded. In particular embodiments the base layer 11 includes visual indications that delimit the marking region 13. This enables indication to the practitioner of a zone of the sterile drape that must not be cut.

The adhesive material 16 enables the optical monitoring device 10 to be glued to the skin of the patient. This adhesive material may be distributed more or less uniformly and continuously or not over the part of the interior face 11a corresponding to the marking region 13.

In the example considered here and depicted in FIG. 2 the adhesive material 16 takes the form of an adhesive tape connecting together the various points at which the optical markers 14 or the fixing supports are situated.

The adhesive tape is for example a polyethylene film coated with an acrylic adhesive or a rayon woven fabric covered with an acrylic adhesive. The use of a rayon woven fabric enables an absorbing character of the base layer 11 in contact with the patient to be preserved.

The adhesive tape may be stitched or glued to the base layer 11. The adhesive tape enables a semi-rigid connection to be made between the optical markers, which facilitates their installation and optimizes their stability. The tape form also makes it possible to minimize the overall size of the marking region 13.

As mentioned above a pre-intervention medical image can represent both the anatomy of interest of the patient and the optical monitoring device 10 to enable determination of the position of the anatomy of interest relative to the optical monitoring device 10.

This image may be for example a medical image obtained by tomodensitometry (or a computed tomography scan (CT-scan)), by angiography or by magnetic resonance imaging (MRI).

In particular embodiments the optical monitoring device 10 includes at least three radio-opaque markers each rigidly fastened to a respective optical marker 14 or to a respective fixing support to assist in determination of the position of the optical monitoring device 10 in the medical image. These radio-opaque markers may for example be ceramic beads or adhesives including a radio-opaque ink.

It should nevertheless be noted that these radio-opaque markers are not necessarily indispensable because the nature of the materials used to manufacture some elements of the optical monitoring device 10 (in particular the optical markers 14) can sometimes enable these elements to be viewed in the medical image.

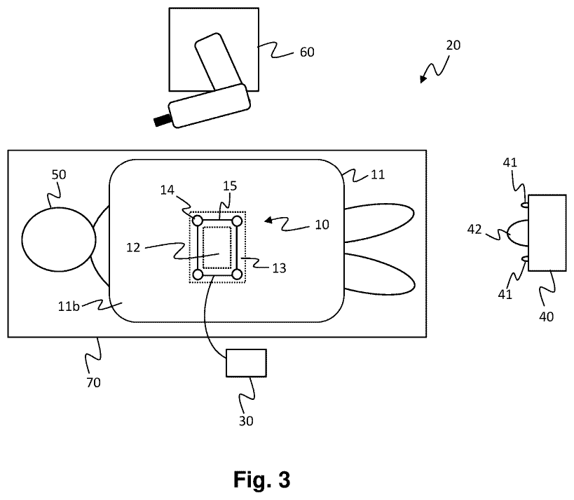

FIG. 3 represents schematically one embodiment of an optical navigation system 20 according to the invention.

The optical navigation system 20 includes an optical monitoring device 10 conforming to any of the embodiments described above. The optical monitoring device 10 is equipped with optical markers 14. In the example depicted in FIG. 3 the optical monitoring device 10 corresponds to that described above with reference to FIGS. 1 and 2.

The optical navigation system 20 also includes a locating device 40 configured to cooperate with the optical markers 14 to determine the position of the optical markers 14 in a frame of reference of the locating device 40.

The locating device 40 includes for example a stereoscopic video camera functioning in the infrared range or in the visible light range. The stereoscopic video camera includes two optical sensors 41 configured to receive light signals from the optical markers 14 and to deduce therefrom the position of the optical markers 14 (on the basis of an arrival time and/or angle of each light signal). These may be light signals transmitted directly by the optical markers 14 when the optical markers 14 are active or light signals reflected by the optical markers 14 when the optical markers 14 are passive. A ToF (time of flight) or RGB-D (red green blue-depth) video camera may be used when the optical markers 14 are active. By way of non-limiting example the Polaris® navigation solution proposed by the company Northern Digital Inc. utilizes a stereoscopic infrared video camera.

The locating device 40 may further include a supplementary video camera 42 for continuous acquisition of images of the intervention. The video camera 42 may in particular be integrated into the stereoscopic video camera system to have the same field of view axis. The video camera 42 may for example be used to monitor the insertion of the medical instrument and to estimate the depth of insertion of the medical instrument at a given time.

In the example considered here and depicted in FIG. 3 a patient 50 is positioned on an intervention table 70 to undergo a minimally-invasive medical intervention on an anatomy of interest. The optical monitoring device 10 is positioned on the skin of the patient 50 near the anatomy of interest. A medical robot 60 is used to assist the practitioner during the intervention. The medical robot includes for example an articulated arm to the end of which is fixed a medical instrument.

The objective of the locating device 40 is to determine in real time the position of the optical markers 14 to deduce therefrom the position of the optical monitoring device 10 in order ultimately to determine the position of the anatomy of interest. The locating device 40 may equally be configured to determine the position of the medical instrument with the aid of another optical monitoring device positioned on the medical robot 60 or directly on the medical instrument. The position of the medical instrument can also be determined with the aid of the video camera 42. When the respective positions of the medical instrument and the anatomy of interest relative to one another are known (for example in a frame of reference of the locating device 40) it is possible to configure the articulated arm of the medical robot 60 to position the medical instrument optimally relative to the anatomy of interest.

The locating device 40 includes for example a control unit including one or more processors configured to determine the respective positions of the anatomy of interest and the medical instrument relative to one another, as well as a communication module configured to transmit that information to the medical robot 60.

The optical navigation system 20 also includes a measuring device 30 configured to cooperate with the fiber optic sensor 15 to determine the relative position of each of the optical markers 14 in a frame of reference of the measuring device 30. The fiber optic sensor 15 is for example connected to the measuring device 30 once the optical monitoring device 10 has been positioned on the patient 50.

The measuring device 30 and the fiber optic sensor 15 form a device that is redundant relative to the locating device 40 for determining the position of the optical markers 14. This is particularly useful when one of more of the optical markers 14 is or are not visible to the locating device 40. This is the case for example when the line of sight between an optical sensor 41 and an optical marker 14 is interrupted by an obstacle (for example the practitioner, an operator, the articulated arm of the medical robot 60, the medical instrument, etc.). When an optical marker 14 is not visible the locating device 40 is not able to determine its position. However if at least one optical marker 14 is visible and can be identified from among all of the optical markers and if the position of each of the optical markers 14 relative to one another is known, then it is possible to deduce therefrom the position of all the optical markers 14.

Thus it is possible to add to the information obtained by the locating device 40 information supplied by the measuring device 30. This enables optimization of the accuracy of the locating device 40 because all of the optical markers 14 can be used permanently.

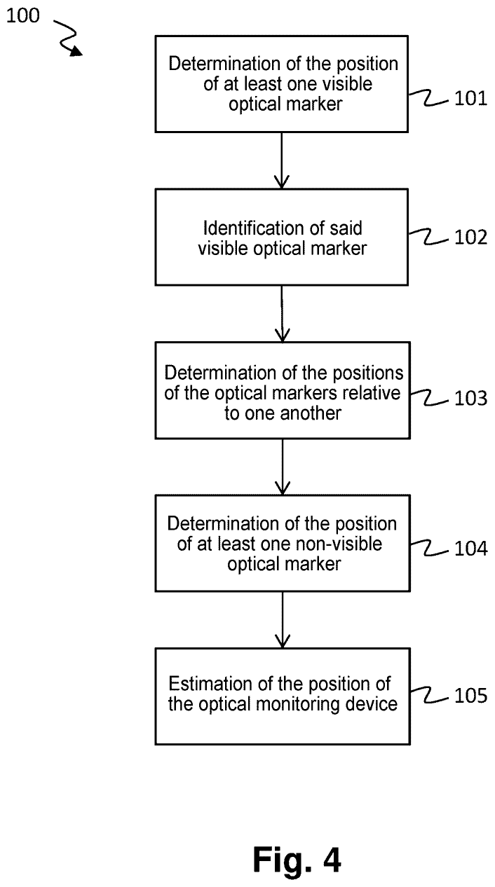

FIG. 4 depicts schematically the main steps of an optical navigation method 100 using a navigation system 20 as described hereinabove with reference to FIG. 3.

The method 100 includes a step 101 of determination with the aid of the locating device 40 of the position of at least one optical marker 14 visible to the locating device 40 (by this is meant that the line of sight between the optical marker 14 and the locating device 40 is not interrupted by an obstacle).

The method 100 then includes a step 102 of identification of said optical marker 14 that is visible from among all of the optical markers 14. As described in detail hereinafter there are various ways to identify an optical marker 14 from among all of the optical markers 14.

The method 100 includes a step 103 of determination with the aid of the measuring device 30 of the positions of all of the optical markers 14 relative to one another.

The method 100 includes a step 104 of determination of the position of at least one optical marker 14 that is not visible to the locating device 40 from the position of the visible optical marker 14 and the positions of the optical markers 14 relative to one another.

Finally, the method 100 includes a step 105 of estimation of the position of the optical monitoring device 10 from the positions of at least three optical markers 14.

There may be various ways to identify an optical marker 14 among all of the optical markers 14.

In a first example, if the optical markers 14 are active, each optical marker 14 can be configured to emit a differently modulated infrared signal. The identification 102 of a visible optical marker 14 can then be effected by identifying the modulation of the infrared signal emitted by said optical marker 14.

In a second example, for passive optical markers 14 there can be envisaged placing at least four optical markers 14 on the optical monitoring device 10 in a particular manner enabling unambiguous identification of the optical markers as soon as at least three optical markers are visible. The optical markers 14 may for example be positioned so that the distances separating two optical markers 14 taken two by two all differ by at least a predetermined margin value (for example, whatever the optical markers in question the distance between two optical markers must differ by at least 5 mm relative to the distance between two other optical markers). When the positions of at least three visible optical markers 14 have been determined by the locating device 40 it is then possible to identify unambiguously said three optical markers 14 from among all of the optical markers 14 on the basis of the distances between two optical markers 14 determined for at least two different pairs of optical markers 14 formed from among said at least three visible optical markers 14.

In a third example the method 100 may include a preliminary step of generating for each optical marker 14 with the aid of the locating device 40 and the measuring device 30 a model representative of a substantially cyclic movement of said optical marker 14. In this case it is possible to identify a visible optical marker 14 by identifying the model corresponding to the observed movement of said visible optical marker 14.

In other words, in this third example the identification 102 of a visible optical marker 14 is effected by using conjointly the movement information supplied by the measuring device and that supplied by the optical locating device 40. The identification 102 of a visible optical marker 14 is determined by analysis of the movement characteristics of each of the optical markers 14, matching the information supplied by the measuring device 30 and that supplied by the optical locating device 40, and the use of those movement characteristics for unambiguous identification of the visible optical marker. Assuming a substantially cyclic movement, as is the case for the movement generated by the breathing of the patient 50, this matching method can enable reconstruction of the full location information of the optical monitoring device 10 even if only one of the optical markers 14 is visible. The movement characteristics of an optical marker 14 may for example correspond to an amplitude of the movement of the optical marker 14 and/or a frequency of movement of the optical marker 14 in a main direction followed by the optical marker 14. These characteristics form a model representative of the movement of the optical marker 14 during a cycle.

The optical navigation method 100 enables significant limitation of the problem of loss of line of sight whilst retaining high accuracy thanks to the conjoint use, on the one hand, of the precise information as to the absolute location in space supplied by the locating device 40 and, on the other hand, the relative position information supplied by the measuring device 30.

In the example depicted in FIG. 3 the optical navigation system 20 may be used in the following manner.

The patient 50 is stopped from breathing or the practitioner asks the patient 50 to hold their breath and the position of the optical markers 14 is recorded (when the patient has been stopped from breathing the position of the optical markers 14 is fixed).

A pre-intervention medical image of the patient 50 is acquired while the patient 50 is not breathing. The pre-intervention medical image enables viewing of both the anatomy of interest of the patient 50 and the optical monitoring device 10. This enables determination of the position of the anatomy of interest relative to the optical monitoring device 10 at the moment in the respiratory cycle in which the patient is not breathing. The knowledge of the position of the optical monitoring device 10 at this moment can then enable determination in real time of the position of the anatomy of interest of the patient at that moment. The patient then begins to breathe normally again.

The pre-intervention medical image is used to plan the surgical intervention. It is for example possible to determine a trajectory that the medical instrument must follow during its insertion. The trajectory is for example defined on the basis of a point of entry at the level of the skin of the patient and a target point at the level of the lesion to be treated.

In order to insert a medical needle the patient is again stopped from breathing at a moment at which the position of the optical monitoring device 10 is substantially the same as the position during the acquisition of the pre-intervention medical image (that is to say substantially in the same phase of the respiratory cycle). This guarantees the anatomy of interest is at substantially the same position as that represented in the pre-intervention medical image used to plan the intervention.

During these operations the practitioner may obstruct the target line of one or more optical markers 14 of the optical monitoring device 10. As soon as at least one optical marker 14 that can be identified from among all of the optical markers 14 is visible to the locating device 40 the position of the optical monitoring device 10 can be determined with good accuracy thanks to the redundancy introduced by the fiber optic sensor 15 and the measuring device 30. The optical navigation system 20 can also make it possible to detect an unexpected movement of the patient.

In some cases it is necessary to insert a plurality of medical instruments (or to insert the same medical instrument more than once) to reach different target points at the level of the lesion. The configuration of the monitoring device 10 makes this possible without any particular constraint as to the point of entry.

The above description clearly shows that because of their various characteristics and advantages the various devices and methods described achieve the objectives set for them. In particular the optical monitoring device 10 is easy to install, has minimum intrusion into the intervention zone, enables optimization of the adhesion to the skin of the patient 50, enables limitation of the area to be disinfected on the skin of the patient 50, and guarantees very precise navigation even if some optical markers 14 are not visible to the locating device 40.

It is to be noted that the embodiments considered hereinabove have been described by way of non-limiting example and that other variants can therefore be envisaged.

In particular the choice of a particular dimension, shape or composition of the base layer 11 of the monitoring device 10 is merely one variant of the invention. The same applies to the choice of a particular shape of the marking region 13 or a particular position of the marking region 13 on the base layer 11. It also applies to the choice of a particular dimension, shape or position of the intervention region 12 on the base layer 11.

As explained hereinabove the optical markers 14 of the monitoring device 10 may equally well be active markers or passive markers.

For the optical navigation method 100 various processes have been presented for identifying at least one visible optical marker 14 from among all of the optical markers 14 of the monitoring device 10. Other processes could nevertheless be used in this said method 100 and the choice of a particular process is merely one variant of the invention.

1. An optical monitoring device comprising a base layer that serves as a sterile drape and has an interior face intended to face skin of a patient and an exterior face opposite the interior face, said base layer comprising an intervention region corresponding to an opening or to a region intended to be cut to expose a zone of the skin of the patient where the intervention is to take place, and a marking region that surrounds at least partially the intervention region, said marking region comprising:
on the interior face, an adhesive material for fixing the optical monitoring device onto the skin of the patient,
on the exterior face, at least three optical markers or at least three fixing supports each intended to receive an optical marker, and
a fiber optic sensor fastened to the base layer and comprising at least one measuring point associated with each of the optical markers or the fixing supports, the fiber optic sensor comprising an optical fiber incorporating Bragg gratings.

2. The optical monitoring device of claim 1, wherein the base layer is a polyethylene film lined with a cellulose absorbent film.

3. The optical monitoring device of claim 1, wherein the intervention region is precut in the base layer.

4. The optical monitoring device of claim 1, wherein the base layer comprises visual indications defining the marking region.

5. The optical monitoring device of claim 1, wherein the adhesive material takes the form of an adhesive tape connecting together the various points at which the optical markers or the fixing supports are situated.

6. The optical monitoring device of claim 5, wherein the adhesive tape is a polyethylene film coated with an acrylic adhesive or a rayon woven fabric covered with an acrylic adhesive.

7. The optical monitoring device of claim 1, comprising at least three radio-opaque markers each rigidly connected to a respective optical marker or a respective fixing support.

8. The optical monitoring device of claim 1, wherein the optical markers are active, each active optical marker being configured to emit a differently modulated infrared signal.

9. The optical monitoring device of claim 1, wherein the optical markers are passive and there are at least four optical markers.

10. An optical navigation system comprising:
an optical monitoring device of claim 1, said optical monitoring device being equipped with optical markers,
a measuring device configured to cooperate with the fiber optic sensor to determine a relative position of each of the optical markers in a frame of reference of the measuring device, and
a locating device configured to cooperate with the optical markers to determine a position of each of the optical markers in a frame of reference of the locating device.

11. An optical navigation method utilizing an optical navigation system of claim 10, said method comprising:
determining with the aid of the locating device of the position of at least one optical marker visible to the locating device,
identifying said at least one visible optical marker from among all the optical markers,
determining with the aid of the measuring device of the positions of all of the optical markers relative to one another,
determining the position of at least one optical marker that is not visible to the locating device on the basis of the position of the visible optical marker and the positions of the optical markers relative to one another, and
estimating the position of the optical monitoring device from the positions of at least three optical markers.

12. The optical navigation method of claim 11, wherein the optical markers are active, each optical marker is configured to emit a differently modulated infrared signal and said at least one visible optical marker is identified by identifying the modulation of the infrared signal emitted by said optical marker.

13. The optical navigation method of claim 11, wherein the optical markers are passive, the optical marking device includes comprises at least four passive optical markers, the distances between two optical markers taken two by two all differ by at least one predetermined margin value, and the method further comprises:
determining with the aid of the locating device of the position of at least three optical markers visible to the locating device, and
identifying said three optical markers from among all of the optical markers on the basis of the distances between two optical markers determined for at least two different pairs of optical markers formed from among said at least three visible optical markers.

14. The optical navigation method of claim 11, further comprising a preliminary step of generating for each optical marker with the aid of the locating device and the measuring device of a model representative of a substantially cyclic movement of said optical marker and in which the identification of said at least one visible optical marker is effected by identifying the model corresponding to the movement of said visible optical marker.
